(19) United States
(12) Patent Application Publication
(10) Pub. No.: US 2022/0127576 A1
(43) Pub. Date: Apr. 28, 2022

(54) COMPOSITIONS AND METHODS FOR STEM CELL THERAPY

(71) Applicants: Arizona Board of Regents on Behalf of the University of Arizona, Tucson, AZ (US); University of Houston System, Houston, TX (US)

(72) Inventors: Richard Simpson, Tucson, AZ (US); Emmanuel Katsanis, Tucson, AZ (US); Richard Bond, Houston, TX (US)

(21) Appl. No.: 17/430,541

(22) PCT Filed: Feb. 12, 2020
(86) PCT No.: PCT/US2020/017928

§ 371 Date: Aug. 12, 2021

Related U.S. Application Data
(60) Provisional application No. 62/804,798, filed on Feb. 13, 2019.

(51) Int. Cl. C12N 5/0789; A61K 35/28; A61K 38/19

(52) U.S. Cl. CPC C12N 5/0647; A61K 38/193; A61K 35/28

(57) ABSTRACT

Provided herein are compositions and methods for improving outcome in stem cell transplantation. In particular, provided herein are compositions and methods for improving efficacy and reducing graft versus host disease in allogenic cell transplantation.

### CROSS-REFERENCE TO RELATED APPLICATION

This application claims priority to and the benefit of U.S. Provisional Application No. 62/804,798, filed Feb. 13, 2019, which is hereby incorporated by reference in its entirety.

### FIELD

Provided herein are compositions and methods for improving outcome in stem cell transplantation. In particular, provided herein are compositions and methods for improving efficacy and reducing graft versus host disease in allogenic cell transplantation.

### BACKGROUND

Allogeneic hematopoietic cell transplantation (alloHCT) offers a cure for many patients with hematologic malignancies, non-malignant blood disorders, primary immunodeficiencies and other conditions (Yang, W. C. et al. Outcome predictors of allogeneic hematopoietic stem cell transplant. Am J Med Sci 345, 33-38 (2013); Mohty, B. & Mohty, M. Long-term complications and side effects after allogeneic hematopoietic stem cell transplantation: an update. Blood cancer journal 1, e16 (2011)). However, alloHCT is unfortunately still associated with significant morbidity and mortality, especially from graft-versus-host disease (GvHD) (Hill, L. et al. New and emerging therapies for acute and chronic graft versus host disease. Ther Adv Hematol 9, 21-46 (2018); Nassereddine, S., Rafei, H., Elbahesh, E. & Tabbara, I. Acute Graft Versus Host Disease: A Comprehensive Review. Anticancer Res 37, 1547-1555 (2017)). This is especially so when G-CSF mobilized peripheral blood hematopoietic cells (PBHCs) are used instead of bone marrow (Nassereddine, S., Rafei, H., Elbahesh, E. & Tabbara, I. Acute Graft Versus Host Disease: A Comprehensive Review. Anticancer Res 37, 1547-1555 (2017); Holtick, U. et al. Comparison of bone marrow versus peripheral blood allogeneic hematopoietic stem cell transplantation for hematological malignancies in adults—a systematic review and meta-analysis. Crit Rev Oncol Hematol 94, 179-188 (2015)). Although several immunosuppressive strategies can reduce the incidence and severity of GvHD (e.g., calcineurin inhibitors, mycophenolate mofetil, methotrexate and post-transplant cyclophosphamide), these are not always effective and can impair engraftment, mitigate the curative graft-versus-tumor (GvT) effect and increase the incidence of opportunistic infections (Hill, L. et al. New and emerging therapies for acute and chronic graft versus host disease. Ther Adv Hematol 9, 21-46 (2018)). There is, therefore, a critical need to improve the composition and function of G-CSF mobilized donor PBHC grafts a priori prior to collection and transplantation.

### SUMMARY

Experiments described herein demonstrated that infusing healthy G-CSF mobilized donors with the non-selective β-adrenergic receptor (β-AR) agonist isoproterenol (ISO) evoked a favorable graft phenotype of mobilized PBHCs without compromising the number of CD34+ HSCs in the graft. Moreover, cytotoxic activity against leukemia and multiple myeloma target cells in vitro was 2-fold greater in the G-CSF+ISO PBHC grafts compared to G-CSF alone. It was found that these effects were mediated through the β₂-AR subtype, as graft composition remained favorably altered in the presence of a selective β₁-AR antagonist (bisoprolol) but not a non-selective β₁+β₂-AR antagonist (nadolol). Accordingly, in some embodiments, provided herein are compositions and methods for mobilizing stem cells with enhanced properties for use in allogenic or autologous transfers.

For example, in some embodiments, provided herein is a method of obtaining stem cells from a donor, comprising: a) administering a stem cell mobilization agent, a (β-adrenergic receptor agonist (β-AR), and at least one additional agent selected from, for example, a phosphodiesterase-4 (PDE-4) inhibitor and a β-AR antagonist; and b) isolating the stem cells from the donor. In some embodiments, the donor is a human. In certain applications, the stem cells are hematopoietic stem cells (e.g., peripheral blood hematopoietic cells).

The present disclosure is not limited to particular β-AR agents. Examples include, but are not limited to, isoproterenol, denopamine, dobutamine, dopexamine, prenalterol, or xamoterol.

The present disclosure is not limited to particular PDE-4 inhibitors. Examples include, but are not limited to, roflumilast, apremilast, cilomilast, crisaborole, diazepam, ibudilast, luteolin, mesembrenone, piclamilast, or rolipram.

The present disclosure is not limited to particular β-AR antagonists (e.g., β₁-AR antagonists). Examples include, but are not limited to, bisoprolol, acebutolol, atenolol, betaxolol, celiprolol, metoprolol, nebivolol, esmolol, propranolol, bucindolol, carteolol, carvedilol, labetalol, nadolol, oxprenolol, penbutolol, pindolol, sotalol, timolol, ccebutolol, butaxamine, or nebivolol. In some embodiments, the β₁-AR antagonist is bisoprolol.

The present disclosure is not limited to particular stem cell mobilization agents. Examples include, but are not limited to, G-CSF, GM-CSF, or plerixafor.

In some embodiments, the additional agent is a PDE-4 inhibitor and a β₁-AR antagonist. In some embodiments, the β₁-AR antagonist and said PDE-4 inhibitor are administered orally. In some embodiments, the β₁-AR antagonist and said PDE-4 inhibitor are administered 1-5 (e.g., 3) hours prior to administration of the β-AR. In some embodiments, the β-AR is administered via infusion for a duration of approximately 4 (e.g., 3-5) hours. In some embodiments, the isolating said stem cells occurs immediately following administration of the β-AR.

In some embodiments, the stem cells exhibit one or more properties selected from, for example, increased graft-versus tumor effect, decreased viral reactivation, reduced graft-versus-host-disease, or reduced lymphoproliferative disease relative to stem cells mobilized by stem cell mobilization agent alone.

Further embodiments provide stem cells isolated by a method as described herein.

Additional embodiments provide a composition comprising stem cells isolated by a method as described herein. In some embodiments, the composition is a pharmaceutical composition.

Certain embodiments provide a composition as described herein for use in administering to a recipient in need thereof. In some embodiments, the recipient has a hematologic malignancy (e.g., leukemia) or an immune system disorder. In some embodiments, the donor is the recipient (e.g., autologous transfer) or a different individual (e.g., allogenic transfer).

Also provided herein is a composition as described herein for use in treating a hematological malignancy or immune disorder.

In some embodiments, provided herein is a method of treating a hematological malignancy or immune system disorder, comprising: administering a composition as described herein to a subject in need thereof.

In additional embodiments, provided herein is the use of a composition as described herein to treat a hematological malignancy or immune system disorder.

In further embodiments, provided herein is a kit for use in preparing a donor for stem cell isolation, comprising one or more of: a stem cell mobilization agent, β-adrenergic receptor agonist (β-AR), a phosphodiesterase-4 (PDE-4) inhibitor, or a β-AR antagonist.

Also provided is the use of the kit for preparing a donor for stem cell isolation.

Additional embodiments are described herein.

### DESCRIPTION OF THE FIGURES

FIG. 1 shows that ISO infusion (50 ng/kg/min) markedly increases the composition of NK-cells and TCR-γδ cells, and simultaneously lowers the composition of CD4+ TCR-αβ cells and B-cells among total blood lymphocytes both before and after 5-days treatment with G-CSF (5 mcg/kg/day). Values are the mean (n=7; 3 females).

FIG. 2A shows that ISO infusion increases the numbers of CD34 HSCs (left panel) and alters the ratio of ‘favorable’ to ‘unfavorable’ lymphocyte subtypes (right panel) in peripheral blood after G-CSF mobilization (left panel) in healthy volunteers.

FIG. 2B shows that effector lymphocytes mobilized to blood with exercise via a catecholamine and β2-AR signaling mechanism, and selective β 1-AR blockade augments NK-cell mobilization with exercise.

FIG. 3 shows the effects of administering bisoprolol+roflumilast on lymphocyte composition in response to systemic β-AR activation. All drug conditions lowered the heart rate response to exercise compared to placebo (right graph). The mobilization of NK-cells (middle graph) and the ratio of ‘favorable’ to ‘unfavorable’ cells in blood was markedly elevated after bisoprolol+roflumilast compared to the resting and placebo condition.

FIG. 4 shows that ISO infusion increases the in vitro anti-tumor activity of G-CSF mobilized PBHC grafts. * indicates significant difference from rest, p<0.05.

FIG. 5 shows that β₁+β₂-AR non-selective blockade (nadolol) but not β₁-AR selective blockade (bisoprolol) inhibits the effects of systemic β-AR activation on the mobilization of VSTs in healthy humans (n=11). Difference from the resting condition indicated by *, p<0.05. Difference from the placebo and bisoprolol trials indicated by #, p<0.05.

FIG. 6A-B: shows that TCR-γδ cells expanded ex vivo with ZOL+IL2 for 14-days after systemic β-AR activation have increased anti-tumor activity against leukemia (K562) and multiple myeloma (U266) target cells in vitro compared to cells expanded at rest and after 1 h of recovery following systemic β-AR activation (A). Antibody blocking experiments in vitro revealed that the augmenting effects against U266 cells are NKG2D dependent (B).

FIG. 7 TCR-γδ cells expanded for 14-days ex vivo after systemic β-AR activation (red bars) have increased anti-tumor activity against leukemia (K562), multiple myeloma (U266) and lymphoma (221.AEH) target cells in vitro compared to cells expanded at rest and after 1 h of recovery following systemic β-AR activation. Data are mean±SEM (n=12).

FIG. 8 shows grafts mobilized with G-CSF+ISO protect against xenoGvHD and weight loss with a trend for improved survival compared to grafts mobilized with G-CSF only.

FIG. 9 shows that lymphocytes mobilized by systemic adrenergic receptor activation (e.g. exercise) have an increased ability to control leukemic growth in vivo.

### DEFINITIONS

To facilitate an understanding of the present disclosure, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, etc.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, and industrial samples. Such examples are not however to be construed as limiting the sample types applicable to the present disclosure.

As used herein, the term “effective amount” refers to the amount of a compound (e.g., a treatment described herein) sufficient to effect beneficial or desired results. An effective amount can be administered in one or more administrations, applications or dosages and is not limited to or intended to be limited to a particular formulation or administration route.

As used herein, the term “co-administration” refers to the administration of at least two agent(s) (e.g., those described herein) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, or ex vivo.

As used herein, the term “toxic” refers to any detrimental or harmful effects on a cell or tissue as compared to the same cell or tissue prior to the administration of the toxicant.

### DETAILED DESCRIPTION OF THE DISCLOSURE

AlloHCT offers a cure for many patients with hematologic malignancies, but GvHD remains a significant cause of morbidity and mortality: AlloHCT is a well-established treatment option for patients with hematologic diseases that cannot be cured with standard therapies. The most common complication of alloHCT is GvHD, a potentially fatal T-cell mediated immune response that targets host tissues and can cause a profound long-lasting state of immunodeficiency in those who survive. Bone marrow was the traditional source for harvesting HSCs, but this has shifted to the use of G-CSF mobilized PBHCs, which is less invasive and results in faster engraftment. Unfortunately, PBHCs are associated with an even greater risk of GvHD compared to bone marrow, which has limited widespread use of PBHCs for alloHCT, particularly in pediatric patients. Thus, new methods are needed to reduce the risk of GvHD using PBHCs without compromising engraftment or the GvT effect.

A ‘favorable’ PBHC graft phenotype exists that is associated with improved patient outcomes as a result of better engraftment, decreased GvHD, infections and/or enhanced GvT: Although several immunosuppressive strategies have been adopted to reduce the incidence and severity of GvHD (e.g. calcineurin inhibitors, mycophenolate mofetil, methotrexate and post-transplant cyclophosphamide), these are not always effective and have adverse effects (Hill, L. et al. New and emerging therapies for acute and chronic graft versus host disease. Ther Adv Hematol 9, 21-46 (2018)). Such agents can impair engraftment, mitigate the GvT effect, and increase the incidence of opportunistic infections. It has emerged that the risk of GvHD and the GvT effects are highly dependent on the cellular composition of the PBHC graft (Table 1) (Gu, G., Yang, J. Z. & Sun, L. X. Correlation of graft immune composition with outcomes after allogeneic stem cell transplantation: Moving towards a perfect transplant. Cell Immunol (2017)). Prospective studies using PBHCs for alloHCT have revealed a ‘favorable’ graft immuno-phenotype preventing GvHD while preserving GvT and engraftment6. This ‘favorable’ immuno-phenotype includes increased proportions of CD34+HSCs, CD56^{dim} NK-cells, TCR-γδ cells, and memory CD8+ TCR-αβ cells, with lower proportions of ‘less favorable’ cell types such as T_{N} TCR-αβ cells, DCs, and CD19+ B-cells. The enrichment of NK-cells and TCR-γδ cells provides viral protection and anti-tumor immunity without causing GvHD, whilst the reduction in TCR-αβ cells (especially T_{N} cells) and B-cells protects against GvHD and posttransplant lymphoproliferative disease, respectively. The ex vivo depletion of TCR-αβ cells and CD19+ B-cells prior to transplant is cumbersome and oftentimes results in graft failure, delayed immune reconstitution, and a greater incidence of infection and relapse (Gu, G., Yang, J. Z. & Sun, L. X. Correlation of graft immune composition with outcomes after allogeneic stem cell transplantation: Moving towards a perfect transplant. Cell Immunol 323, 1-8 (2018); Ciurea, S. O. et al. Improved early outcomes using a T cell replete graft compared with T cell depleted haploidentical hematopoietic stem cell transplantation. Biol Blood Marrow Transplant 18, 1835-1844 (2012)). There is, therefore, a need to find new methods for augmenting the cellular composition of T-cell replete PBHC grafts by enriching with lymphoid cells that have known GvT and anti-viral activity concurrently with favorable GvHD effects.

Pharmacologically targeting the β₂-AR in vivo is used to favorably alter the composition and, consequently the function, of donor PBHC grafts prior to collection and transplant, leads to the development of better therapeutic agents to generate superior PBHC grafts for alloHSCT (Michel, M. C., Seifert, R. & Bond, R. A. Dynamic bias and its implications for GPCR drug discovery. Nat Rev Drug Discov 13, 869 (2014)).

The described compositions and methods for altering the phenotype and function of donor PBHC grafts in vivo for alloHCT are simple, economical and can be universally applied; reducing or eliminating the need for complex ex vivo cell engineering methods, which are costly, labor intensive and limited to larger transplant centers.

The described methods reduce GvHD and increase both anti-tumor and anti-viral activity of the graft in a one-step approach. Increasing the potency and safety of PBHC grafts for alloHSCT may increase their use, particularly in pediatric patients who still rely predominantly on bone marrow sources.

To reduce the risk of GvHD, the number of CD34+ HSCs transplanted is oftentimes curtailed based on the number of concomitant T-cells in the PBHC graft. The described methods, by lowering the number of alloreactive/GvHD causing T-cells in the PBHC graft, allow for the transplantation of more CD34+ HSCs, ensuing better engraftment with fewer complications.

The methods can also be applied to donor lymphocyte infusions (DLI) without G-CSF as a relapse prophylactic/treatment in the post-transplant stage.

The described compositions and methods therefore fulfill an unmet clinical need—no methods currently exist to prep G-CSF mobilized donors to produce superior PBHC grafts in vivo prior to collection and transplant.

For example, in some embodiments, provided herein is a method of mobilizing and obtaining stem cells from a donor, comprising administering a stem cell mobilization agent, a β-adrenergic receptor agonist (β-AR), and at least one additional agent selected from, for example, a phosphodiesterase-4 (PDE-4) inhibitor and a β-AR antagonist, followed by isolating the stem cells from the donor.

In some embodiments, stem cells (e.g., found in PBMCs) are further purified following isolation of blood. PBMCs are made up of lymphocytes (B cells, T cells and NK cells), monocytes and dendritic cells. Density centrifugation (Ficoll-Paque) is the most typical method to isolate PBMCs. Other isolation methods are described, for example, in Grievink et al. (Biopresery Biobank. 2016 October; 14(5):410-415. Epub 2016 Apr. 22); herein incorporated by reference in its entirety.

In some embodiments, the donor is a human. In certain applications, the stem cells are hematopoietic stem cells (e.g., peripheral blood hematopoietic cells). The compositions and methods described herein find use in both autologous and allogenic transplantation methods.

The present disclosure is not limited to particular stem cell mobilization agents. Examples include, but are not limited to, G-CSF, GM-CSF, or plerixafor.

The present disclosure is not limited to particular β-AR agents. Examples include, but are not limited to, isoproterenol, denopamine, dobutamine, dopexamine, prenalterol, or xamoterol.

The present disclosure is not limited to particular PDE-4 inhibitors. Examples include, but are not limited to, roflumilast, apremilast, cilomilast, crisaborole, diazepam, ibudilast, luteolin, mesembrenone, piclamilast, or rolipram.

The present disclosure is not limited to particular β-AR antagonists (e.g., β₁-AR antagonists). Examples include, but are not limited to, bisoprolol, acebutolol, atenolol, betaxolol, celiprolol, metoprolol, nebivolol, esmolol, propranolol, bucindolol, carteolol, carvedilol, labetalol, nadolol, oxprenolol, penbutolol, pindolol, sotalol, timolol, ccebutolol, butaxamine, or nebivolol. In some embodiments, the β₁-AR antagonist is bisoprolol.

In some embodiments, the additional agent is a PDE-4 inhibitor and/or a β₁-AR antagonist. As described in Example 2, in some embodiments, Gs signaling downstream of the β₂-AR (directed Gs signaling) is enhanced (Hatzelmann, A. & Schudt, C. Anti-inflammatory and immunomodulatory potential of the novel PDE4 inhibitor roflumilast in vitro. J Pharmacol Exp Ther 297, 267-279 (2001)). Alternatively, targeting the β-arrestin pathway is achieved by administering the β-arrestin ERK1/2 biased ligand carvedilol, which augments β-arrestin activation and simultaneously blocks signaling via the β₁-AR and the Gs pathway (directed β-arrestin signaling) (Kumari, P. et al. Functional competence of a partially engaged GPCR-beta-arrestin complex. Nature communications 7, 13416 (2016); Wisler, J. W. et al. A unique mechanism of beta-blocker action: carvedilol stimulates beta-arrestin signaling. Proc Natl Acad Sci USA 104, 16657-16662 (2007)). Directing ISO or comparable agent signaling through either of these pathways with the chosen pharmaceuticals involves blocking the β₁-AR. This is highly advantageous for two reasons: (i) it increases ISO availability for the preferred target (β₂-AR) thus augmenting signaling via this receptor; and (ii) transient mild tachycardia (in the order of 15 to 20 beats/min) and hypertension (in the order of 10 to 20 mmHg) during ISO infusion is greatly diminished as these are largely β₁-AR mediated responses (Arnold, J. M. et al. Effects of the beta 2-adrenoceptor antagonist ICI 118,551 on exercise tachycardia and isoprenaline-induced beta-adrenoceptor responses in man. British journal of clinical pharmacology 19, 619-630 (1985); Ladage, D., Schwinger, R. H. & Brixius, K. Cardio-selective beta-blocker: pharmacological evidence and their influence on exercise capacity. Cardiovasc Ther 31, 76-83 (2013)).

In some embodiments, the β₁-AR antagonist and/or PDE-4 inhibitor are administered orally. In some embodiments, the β₁-AR antagonist and said PDE-4 inhibitor are administered 1-5 (e.g., 3) hours prior to administration of the β-AR. In some embodiments, the β-AR is administered via infusion for a duration of approximately 4 (e.g., 3-5) hours. In some embodiments, the isolating of stem cells occurs immediately following administration of the β-AR.

In some embodiments, as described above, the stem cells isolated using the methods described herein exhibit one or more properties selected from, for example, increased graft-versus tumor effect, decreased viral reactivation, reduced graft-versus-host-disease, or reduced lymphoproliferative disease relative to stem cells mobilized by stem cell mobilization agent alone.

Additional embodiments provide a composition comprising stem cells isolated by a method as described herein. In some embodiments, the composition is a pharmaceutical composition.

The cells mobilized and isolated using the methods described herein find use in the treatment of any number of conditions, including, but not limited to, a hematological malignancy (e.g., leukemia) or immune disorder.

In further embodiments, provided herein is a kit for use in preparing a donor for stem cell isolation, comprising one or more of: a stem cell mobilization agent, β-adrenergic receptor agonist (β-AR), a phosphodiesterase-4 (PDE-4) inhibitor, or a β-AR antagonist.

The present disclosure further provides pharmaceutical compositions (e.g., comprising the compounds described above for use in mobilizing stem cells). The pharmaceutical compositions of the present disclosure may be administered in a number of ways depending upon the composition. While exemplary methods are described above, the present disclosure is not limited to a particular administration method.

Administration may be pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

The pharmaceutical formulations of the present disclosure, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present disclosure may be formulated into any of many possible dosage forms such as, but not limited to, tablets, capsules, liquid syrups, soft gels, suppositories, and enemas. The compositions of the present disclosure may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

The compositions of the present disclosure may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on the patient and application. Exemplary, non-limiting dosage regimens are described in the Experimental section below. Optimal dosing schedules can be calculated from measurements of drug accumulation in the body of the patient. The administering physician can easily determine optimum dosages, dosing methodologies and repetition rates. Optimum dosages may vary depending on the relative potency of individual agents, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the oligonucleotide is administered in maintenance doses, ranging from 0.01 μg to 100 g per kg of body weight, once or more daily, to once every 20 years.

### EXPERIMENTAL

The following examples are provided in order to demonstrate and further illustrate certain preferred embodiments and aspects of the present disclosure and are not to be construed as limiting the scope thereof.

### Example 1

### ISO Mobilized Grafts

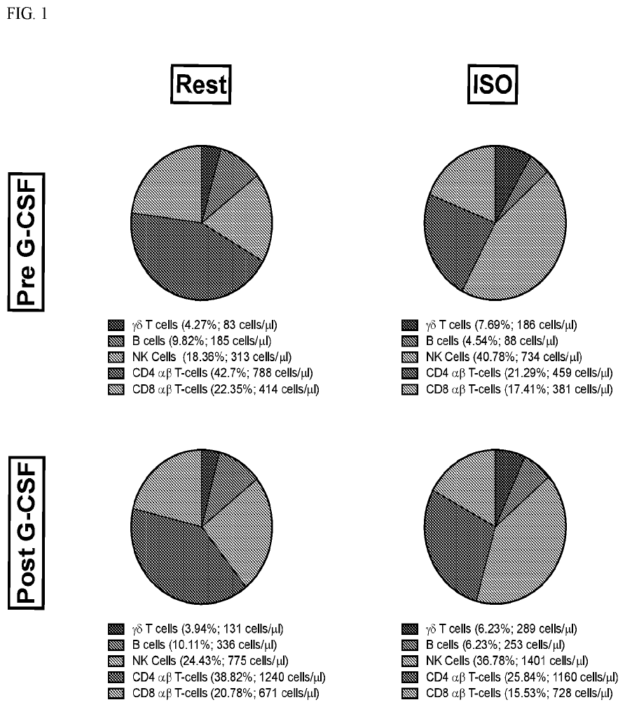

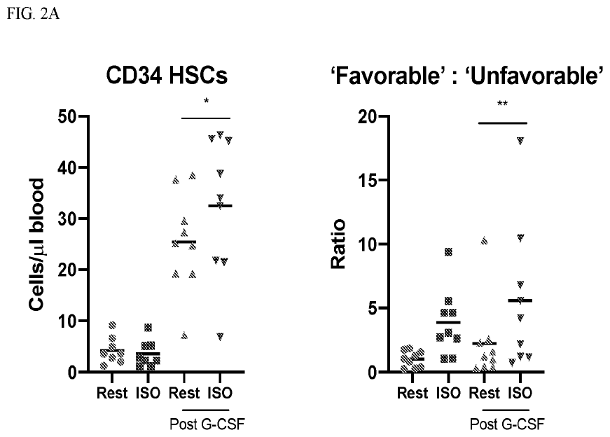

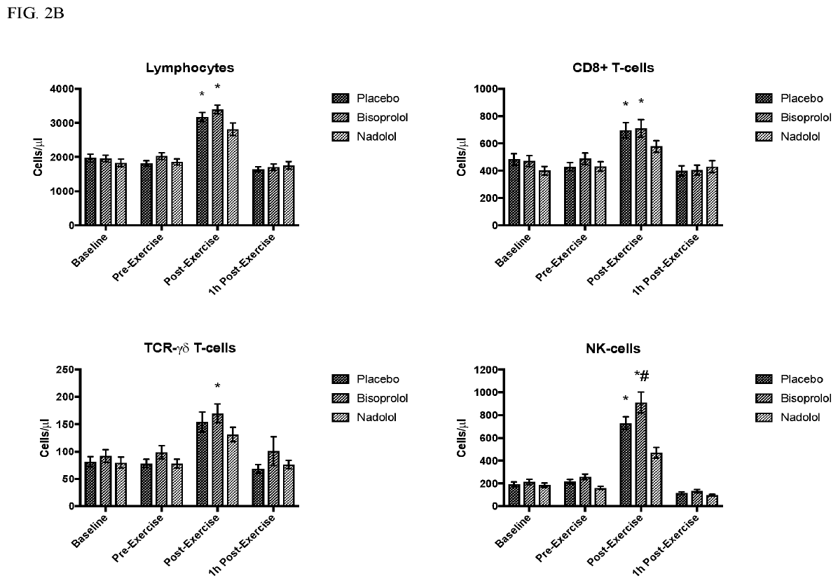

Infusing G-CSF mobilized healthy volunteers with ISO was shown to result in marked alterations in the cellular composition of the graft without compromising the total number of CD34+ HSCs (Table 2; FIG. 1, 2A). Specifically, it was found that G-CSF+ISO mobilized grafts have many more NK-cells, TCR-γδ cells, viral-specific T-cells (VSTs) and much fewer naïve CD4+ TCR-αβ cells and CD19+ B-cells compared to grafts mobilized with G-CSF alone. This graft phenotype is predictive of improved clinical outcomes post alloHSCT, including lower incidence/severity of GvHD and viral infections, better engraftment and a lower incidence of relapse (Table 1). FIG. 2A shows that ISO infusion increases the numbers of CD34 HSCs (left panel) and alters the ratio of ‘favorable’ to ‘unfavorable’ lymphocyte subtypes (right panel) in peripheral blood after G-CSF mobilization (left panel) in healthy volunteers.

FIG. 2B shows that effector lymphocytes are mobilized to blood with exercise via a catecholamine and β2-AR signaling mechanism, and selective β 1-AR blockade augments NK-cell mobilization with exercise. Baseline samples were collected before drug/placebo administration and again 3 h later (pre-exercise). Participants then completed 30-minutes of steady state cycling exercise with blood collected immediately post and 1 h post-exercise. * indicates significant difference from nadolol; # indicates significant difference from placebo, p<0.05 (n=18).

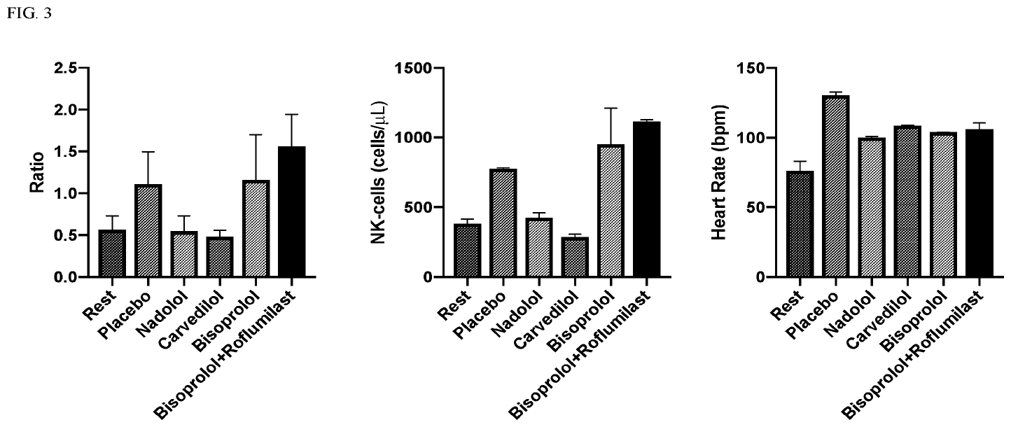

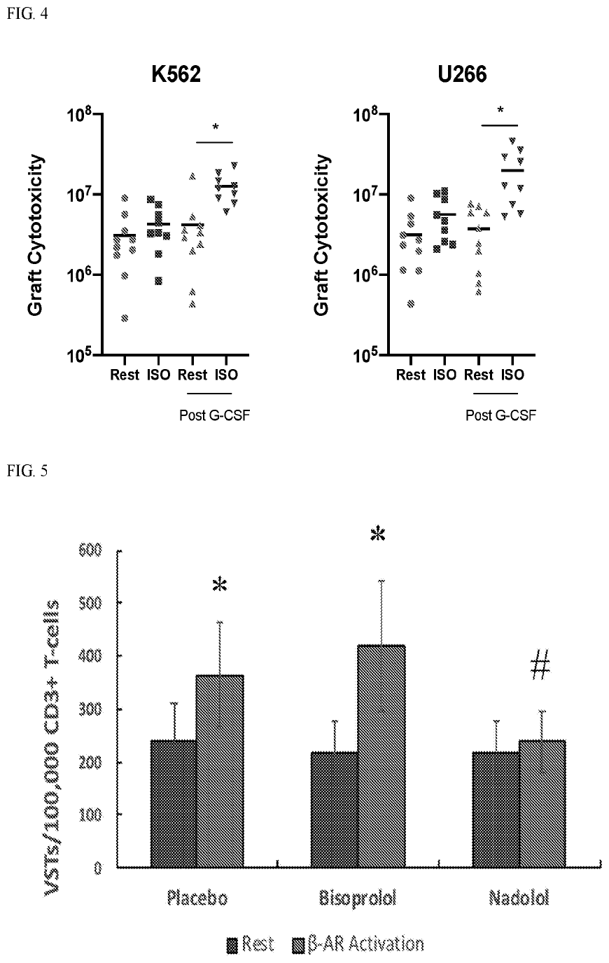

To determine the β-AR subtype involved, a selective β₁-AR antagonist (bisoprolol) and non-selective β₁+β₂-AR antagonist (nadolol) were administered prior to systemic β-AR activation (using dynamic exercise as an experimental model). Nadolol abrogated the effects of systemic β-AR activation on graft composition shifts whereas bisoprolol did not (FIG. 3), revealing that methods of systemic β-AR activation (e.g. ISO infusion, exercise) enrich donor allografts of favorable immune cell subtypes via the β₂-AR.

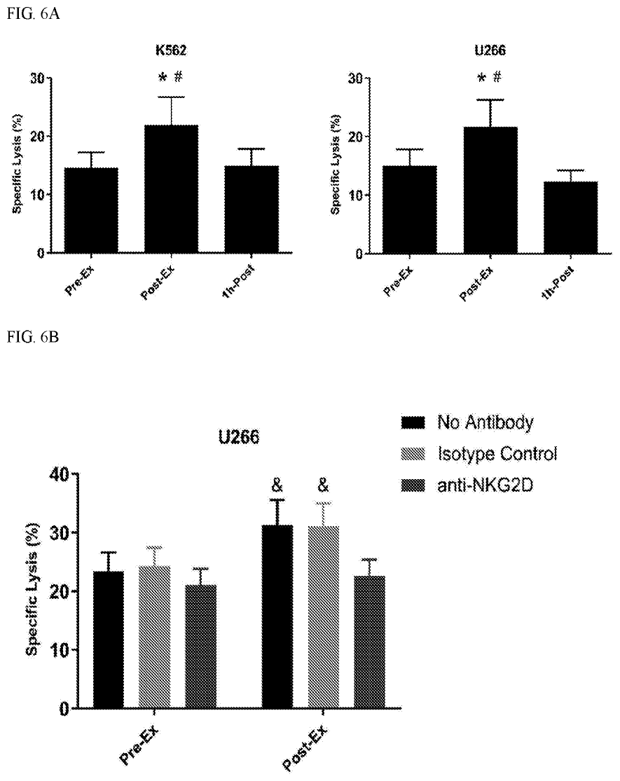

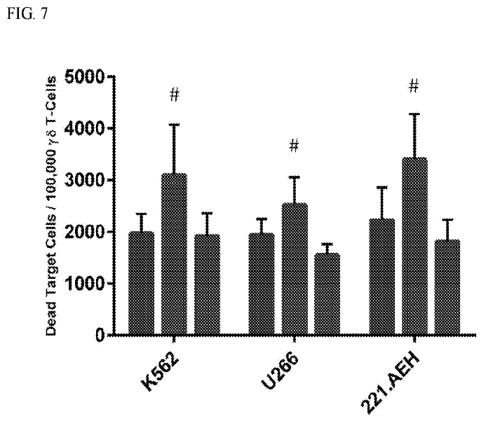

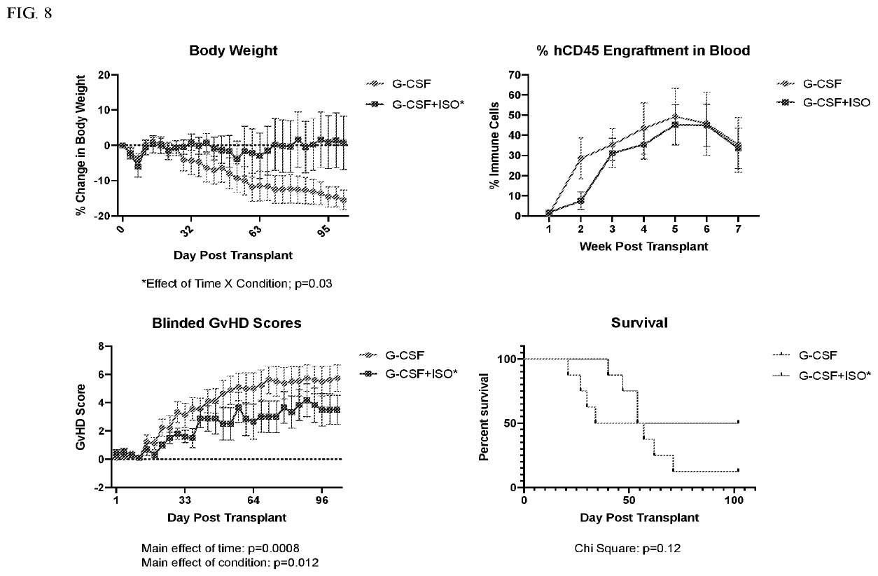

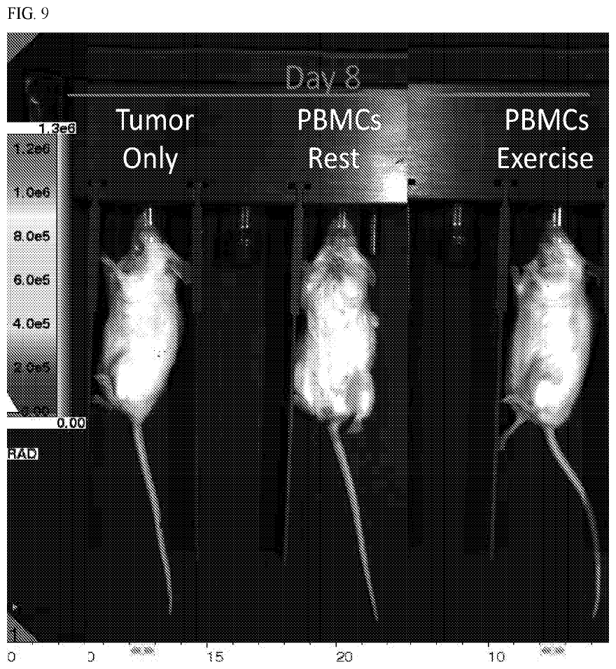

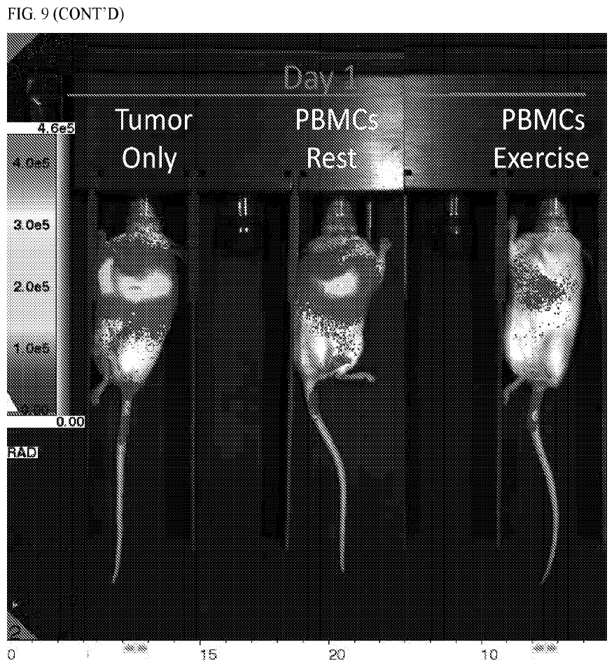

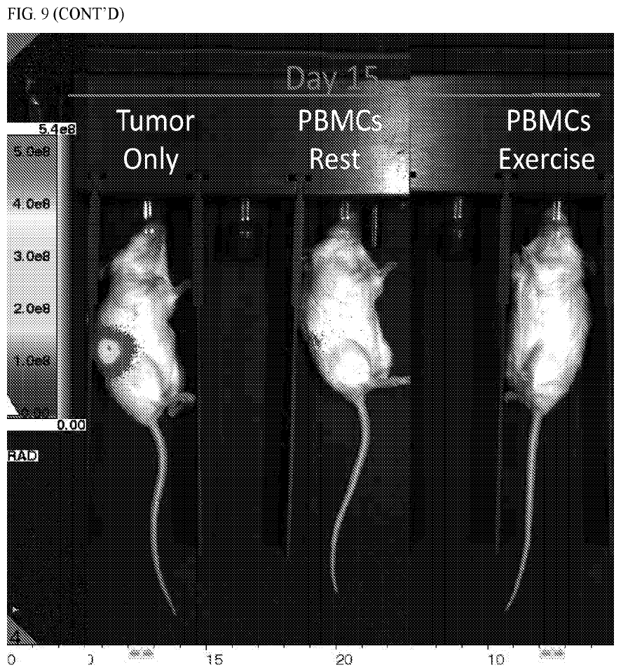

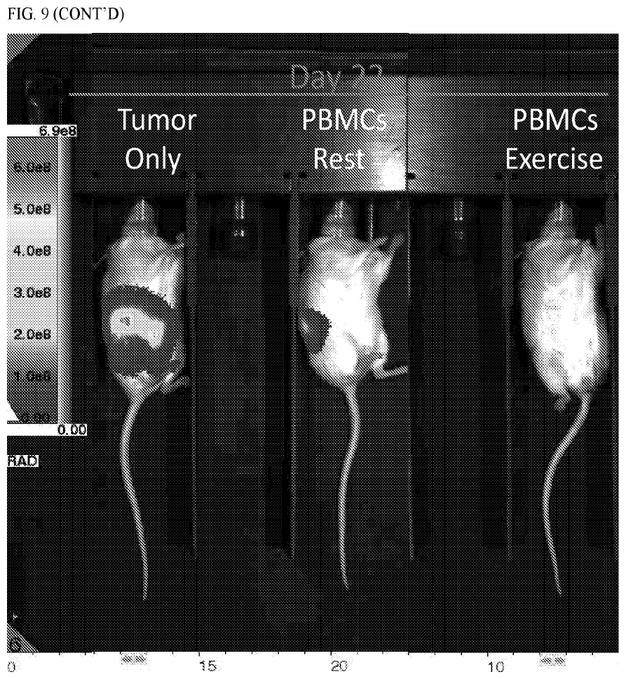

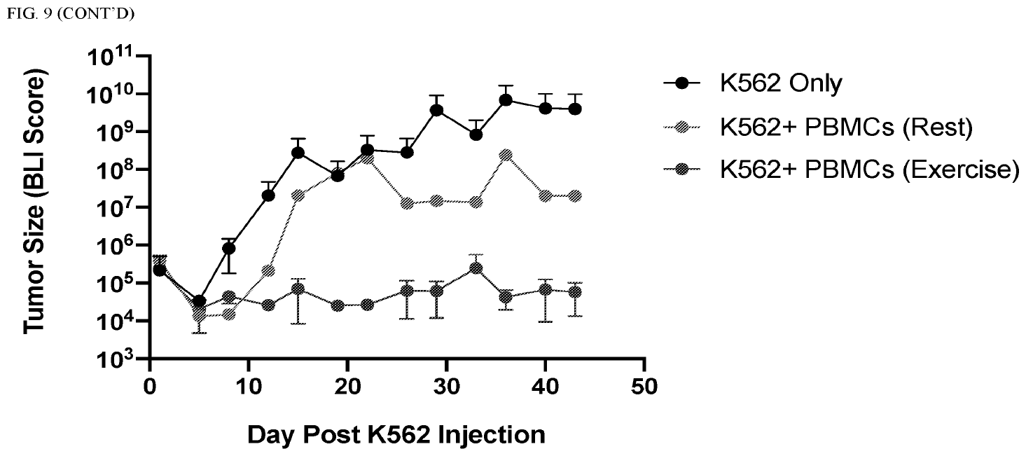

The total in vitro anti-tumor activity of G-CSF+ISO mobilized PBHC grafts was markedly greater than PBHC grafts obtained with G-CSF only (FIG. 3). This was largely explained by a shift in the composition of NK-cells and TCR-γδ cells. Moreover, using both IFN-γ ELISpot and IFN-γ capture assays, it was found that greater numbers of VSTs specific to CMV (pp65 and IE-1), EBV (BMLF-1 and LMP-2) and adenovirus (hexon and penton) antigens were present in PBMCs mobilized with systemic β-AR activation; an effect that was blocked with nadolol but not bisoprolol (FIG. 5). The ex vivo activation and expansion of VSTs to viral peptides+growth cytokines and of TCR-γδ cells to ZOL+IL-2 stimulation was also markedly elevated with systemic β-AR activation (FIG. 6). The cytotoxicity of the NK-cells (not shown) and TCR-γδ cells remained elevated after several weeks of culture (FIG. 7), indicating that the effects of systemic β-AR activation are likely to have long-lasting effects that will benefit the patient following transplantation. The effects of systemic β-AR activation on the mobilization and ex vivo expansion of VSTs and TCR-γδ cells were β₂-AR mediated as the effects were diminished by administering nadolol but not bisoprolol prior to systemic β-AR activation (FIG. 6). FIG. 8 shows grafts mobilized with G-CSF+ISO protect against xenoGvHD and weight loss with a trend for improved survival compared to grafts mobilized with G-CSF only. FIG. 9 shows that lymphocytes mobilized by systemic adrenergic receptor activation (e.g. exercise) have an increased ability to control leukemic growth in vivo.

TABLE 1
The immune cell subtypes found in donor PBHC grafts that are associated
with ‘favorable’ (Top), ‘unfavorable’ (Bottom) and both ‘favorable’
and ‘unfavorable’ (Middle) clinical outcomes in patients after alloHSCT.

| Cell Type | Phenotype | Clinical Outcome |
|---|---|---|
| NK-cells | CD3−/CD56+ | Decreased relapse |
| | | Improved survival |
| TCR-γδ cells | CD3+/CD4−/CD8−/ | Lower Infections, increased event- |
| | TCR-γδ+/TCR-αβ− | free survival |
| NK T-cells | CD3+/CD56+ | Improved disease-free-survival |
| Memory CD8+ | CD3+/CD4−/CD8+/ | Better survival, decreased relapse |
| TCR-αβ cells | TCR-αβ+/CD62L−/ | |
| | CD45RA+/− | Rapid platelet recovery |
| | | Viral protection |
| HSCs | CD34+ and/or | Better engraftment |
| | CD133+ | |
| Activated | CD3−/CD56+/ | Lower aGvHD |
| NK-cells | CD158b+/ | |
| | CD69+ | |
| Regulatory | CD4+/CD25^{hi}/ | Improved GvHD outcomes |
| T-cells (T_{regs}) | CD127^{dim/neg} | |
| Monocytic | CD11b+/CD33+/ | Lower aGvHD |
| MDSCs | CD14+/ | |
| | HLA-DR−/lo | |
| Granulocytic | CD11b+/CD33+/ | Lower aGvHD |
| MDSCs | CD15+/ | |
| | CD66b+/HLA- | |
| | DR−/lo | |
| Viral-Specific | Tetramer+ or IFN− | Lower incidence and severity of |
| T-cells (VSTs) | γ+ to viral peptide | post-transplant viral infections |
| | stimulation | |
| Invariant NK | CD3+/iNKT+ | Improved GVHD, progression-free |
| T-cells | | surviva, Decreased disease-free- |
| | | survival and overall survival |
| Naïve CD4+ | CD3+/CD4+ or | Increased incidence and severity |
| and/or CD8+ | CD8+/CD45RA+/ | of aGvHD and cGvHD |
| TCR-αβ cells | CD62L+ | |
| Dendritic Cells | CD11c+ | Higher risk of relapse |
| | | Increased risk of aGvHD |
| Activated | CD19+/CD20+/ | Higher risk of cGvHD |
| B-cells | CD23+/CD25+ | |

‘Favorable’: Associated with better patient outcomes if elevated in the donor PBHC graft
‘Unfavorable’: Associated with poorer patient outcomes if elevated in the donor PBHC graft
aGvHD: acute GvHD
cGvHD: chronic GvHD

TABLE 2
Absolute (cells/ul) number of lymphocytes, lymphocyte
subtypes and CD34+ HSCs in whole blood at rest and
during ISO infusion before and after G-CSF treatment
(n = 3; 1 female). Values are mean (range).

| | Pre G-CSF | | Post G-CSF | |
|---|---|---|---|---|
| | Rest | ISO | Rest | ISO |
| Lymph. | 1810 | 2043 | 5866 | 6730 |
| | (1680-2032) | (1700-2220) | (4980-7290) | (5860-7580) |
| NK | 254 | 778 | 1158 | 2068 |
| cells | (192-310) | (616-1085) | (747-1941) | (1835-2212) |
| γδ | 39 | 91 | 93 | 214 |
| T cells | (16-78) | (33-160) | (45-165) | (60-341) |
| αβ | 999 | 830 | 2661 | 2253 |
| T cells | (880-1139) | (679-1022) | (2361-2935) | (2253-2968) |
| B cells | 153 | 100 | 456 | 392 |
| | (73-244) | (40-199) | (320-635) | (196-645) |
| CD3⁺ | 1039 | 924 | 2755 | 2933 |
| T cells | (914-1208) | (764-1183) | (2432-3101) | (2500-3311) |
| CD4⁺ | 677 | 489 | 1749 | 1835 |
| T cells | (550-809) | (368-717) | (1497-1917) | (1356-2084) |
| Naïve | 385 | 257 | 860 | 886 |
| | (239-461) | (142-380) | (171-1209) | (36-1422) |
| CM | 179 | 137 | 268 | 298 |
| | (264) | (75-247) | (42-466) | (10-554) |
| EM | 98 | 80 | 405 | 429 |
| | (53-159) | (37-115) | (220-747) | (251-752) |
| EMRA | 14 | 13 | 216 | 220 |
| | (1-33) | (0.71-34) | (30-535) | (23-556) |
| CD8⁺ | 298 | 329 | 786 | 795 |
| T cells | (292-300) | (295-391) | (749-820) | (740-871) |
| Naïve | 136 | 99 | 302 | 227 |
| | (116-170) | (69-134) | (135-412) | (25-357) |
| CM | 35 | 34 | 47 | 41 |
| | (14-65) | (16-67) | (8-105) | (2-102) |
| EM | 73 | 114 | 284 | 342 |
| | (62-86) | (95-125) | (261-323) | (205-502) |
| EMRA | 52 | 81 | 154 | 184 |
| | (2-83) | (3-158) | (22-321) | (19-341) |
| CD34⁺ | 6 | 6 | 110 | 92 |
| HSCs | (3-9) | (1-13) | (72-134) | (61-120) |

### Example 2

### Signally Pathways

β₂-AR signaling can activate at least two pathways; (i) the canonical Gs-cAMP pathway, and (ii) a β-arrestin-dependent pathway (Michel, M. C., Seifert, R. & Bond, R. A. Dynamic bias and its implications for GPCR drug discovery. Nat Rev Drug Discov 13, 869 (2014)). Experiments are conducted to identify the signaling pathway (Gs versus β-arrestin) by which ISO improves the composition and function of donor PBHC grafts. This provides one of three possible pharmaceutical graft engineering strategies for altering donor graft composition and minimizing transient tachycardia and hypertension during ISO infusion for the subsequent aims: (i) Gs pathway directed (bisoprolol+roflumilast+ISO); (ii) β-arrestin pathway directed (carvedilol+ISO); or (iii) non-directed (bisoprolol+ISO) β₂-AR signaling. The response to carvedilol is compared to that of nadolol—a ‘non-selective’ β₁+β₂-AR antagonist that blocks both the Gs and the β-arrestin pathway. If carvedilol and nadolol both inhibit ISO-induced changes in donor graft composition then this indicates involvement of the Gs pathway. Involvement of the Gs pathway is confirmed if bisoprolol and roflumilast cause further alterations in graft composition compared to bisoprolol alone. Conversely, if nadolol but not carvedilol inhibits ISO-induced changes in graft composition, this indicates involvement of the β-arrestin pathway. The effects of a blinded saline infusion and ISO with an orally administered placebo are used as controls. It is further determined if donor comfort and tolerance to prolonged ISO infusion for up to 4 h when administered with the optimal pharmaceutical graft engineering strategy identified above is present.

The below Table provides experimental design for this study.

| | Visits 2-7 (randomized, double-blind, complete | | | |
|---|---|---|---|---|
| Visit 1 | cross-over design, 7-day wash out) | | | |
| Eligibility | Oral Drug: Single Dose | | Infusion: Administered at | |
| Criteria | Administered at 0 h | | 3 h for 30-minutes | |
| Determined | 1. | Placebo | 1. | Placebo (saline) |
| | 2. | Placebo | 2. | ISO (50 ng/kg/min) |
| | 3. | Nadolol (80 mg) | 3. | ISO (50 ng/kg/min) |
| | 4. | Bisoprolol (10 mg) | 4. | ISO (50 ng/kg/min) |
| | 5. | Carvedilol (25 mg) | 5. | ISO (50 ng/kg/min) |
| | 6. | Roflumilast (0.5 mg) + | 6. | ISO (50 ng/kg/min) |
| | | Bisoprolol (10 mg) | | |

| | | | |
|---|---|---|---|
| −7 Days | 0 h | 3 h | +3.5 h |
| blood | blood | blood | blood |

Research Design: Scientific Rigor is maintained through the use of a randomized, complete cross-over, double blind placebo controlled experimental design (above table). This design allows participants to act as their own controls, thus minimizing variability and the impact of potential between-subject confounders. Healthy volunteers report to the lab on six separate occasions at the same time of day. During Visit 1, they undergo an extensive screening procedure to ensure that they meet the study inclusion/exclusion criteria. A blood sample is drawn to determine CMV serostatus (positive or negative) and MR haplotype (A or B), which is included as co-variates in the analysis. Participants then complete six trials on separate days (Visit 2-7) with 7-days interspersed between each trial. During each of these visits, subjects are infused with 50 ng/kg/min of ISO (5 visits) or saline (1 visit) for 30-minutes. Blood samples are drawn before and during the last 10-minutes of the 30-minute infusion protocol. Participants are given orally administered drugs or placebo as a single dose within a few hours prior to ISO infusion. Participants are assigned to trial order using a block randomization scheme with random block sizes of between 2 and 4 subjects to obtain a balanced sample of 8 subjects per trial sequence order (see Statistical Power and Analysis section below). The randomization list is used to create sealed envelopes that are opened to determine assignment to drug order group after visit 1, with the trial order blinded to both the investigators and the participants. A 7-day wash out period and administration of the oral drug/placebo 3 h before infusion because peak serum concentrations of bisoprolol, carvedilol, nadolol and roflumilast occur 1.5-4 h after oral administration, with a half-life of 10-12 h, 7-10 h, 14-24 h and 15-18 h respectively is used (Le Coz, F. et al. Oral pharmacokinetics of bisoprolol in resting and exercising healthy volunteers. Journal of cardiovascular pharmacology 18, 28-34 (1991); Leopold, G., Pabst, J., Ungethum, W. & Buhring, K. U. Basic pharmacokinetics of bisoprolol, a new highly beta 1-selective adrenoceptor antagonist. Journal of clinical pharmacology 26, 616-621 (1986); Schafer-Korting, M., Bach, N., Knauf, H. & Mutschler, E. Pharmacokinetics of nadolol in healthy subjects. European journal of clinical pharmacology 26, 125-127 (1984); Kostis, J. B., Lacy, C. R., Krieger, S. D. & Cosgrove, N. M. Atenolol, nadolol, and pindolol in angina pectoris on effort: effect of pharmacokinetics. American heart journal 108, 1131-1136 (1984); Morgan, T., Anderson, A., Cripps, J. & Adam, W. Pharmacokinetics of carvedilol in older and younger patients. Journal of human hypertension 4, 709-715 (1990)). An ISO dose of 50 ng/kg/min is used as this has been shown to be safe for infusing healthy subjects, causing only mild elevations in systolic blood pressure and heart rate, yet a robust mobilization of lymphocytes to the circulation (Anane, L. H. et al. Mobilization of gammadelta T lymphocytes in response to psychological stress, exercise, and beta-agonist infusion. Brain Behav Immun 23, 823-829 (2009); Mills, P. J., Farag, N. H., Perez, C. & Dimsdale, J. E. Peripheral blood mononuclear cell CD62L and CD11a expression and soluble interstitial cell adhesion molecule-1 levels following infused isoproterenol in hypertension. Journal of hypertension 20, 311-316 (2002); Mills, P. J., Goebel, M., Rehman, J., Irwin, M. R. & Maisel, A. S. Leukocyte adhesion molecule expression and T cell naive/memory status following isoproterenol infusion. J Neuroimmunol 102, 137-144 (2000); Mills, P. J., Karnik, R. S. & Dillon, E. L-selectin expression affects T-cell circulation following isoproterenol infusion in humans. Brain Behav Immun 11, 333-342 (1997)). The subjects (n=3; 1 female) in Example 1 demonstrated excellent tolerance to ISO infusion at this dose, both before and after 5-days of G-CSF treatment (5 mcg/kg/day) with tachycardia and systolic hypertension in the order of ˜50 bpm and ˜30 mmHg, respectively. Relevant Biological Variables: Equal numbers of male and female subjects of any race are recruited. All participants are between 21 and 44 years of age and considered healthy enough to participate in moderate to vigorous intensity exercise (deemed similar to the effects of acute ISO infusion) in accordance with the guidelines published by the ACSM/AHA (Riebe, D. et al. Updating ACSM's Recommendations for Exercise Preparticipation Health Screening. Med Sci Sports Exerc 47, 2473-2479 (2015)). Strict adherence to pre-test guidelines is implemented as described in previous publications (Bigley, A. B. et al. Acute exercise preferentially redeploys NK-cells with a highly-differentiated phenotype and augments cytotoxicity against lymphoma and multiple myeloma target cells. Brain Behav Immun 39, 160-171 (2014); Spielmann, G. et al. The effects of age and latent cytomegalovirus infection on the redeployment of CD8+ T cell subsets in response to acute exercise in humans. Brain Behav Immun 39, 142-151 (2014); Spielmann, G., Bollard, C. M., Kunz, H., Hanley, P. J. & Simpson, R. J. A single exercise bout enhances the manufacture of viral-specific T-cells from healthy donors: implications for allogeneic adoptive transfer immunotherapy. Scientific reports 6, 25852 (2016); Agha, N. H. et al. Vigorous exercise mobilizes CD34+ hematopoietic stem cells to peripheral blood via the beta2-adrenergic receptor. Brain Behav Immun 68, 66-75 (2018)). Outcome measures are adjusted for age (continuous variable), sex (male or female), CMV IgG serostatus (positive or negative) and NK-cell MR gene haplotype (Group A or Group B). These variables are incorporated into the analyses as it was previously shown that CMV serostatus has a major impact on lymphoid composition in response to systemic β-AR activation. Specifically, CMV seropositive individuals mobilizing more CD8+ T-cells but less NK-cells than those who are CMV seronegative (Spielmann, G. et al. The effects of age and latent cytomegalovirus infection on the redeployment of CD8+ T cell subsets in response to acute exercise in humans. Brain Behav Immun 39, 142-151 (2014); Bigley, A. B. et al. NK-cells have an impaired response to acute exercise and a lower expression of the inhibitory receptors KLRG1 and CD158a in humans with latent cytomegalovirus infection. Brain Behav Immun 26, 177-186 (2012); Bigley, A. B. et al. Acute exercise preferentially redeploys NK-cells with a highly-differentiated phenotype and augments cytotoxicity against lymphoma and multiple myeloma target cells. Part II: impact of latent cytomegalovirus infection and catecholamine sensitivity. Brain Behav Immun 49, 59-65 (2015)). Similarly, it has been shown that the magnitude of NK-cell mobilization to the blood following systemic β-AR activation is influenced by the MR-expression profile on NK-cells (Bigley, A. B. et al. Acute exercise preferentially redeploys NK-cells with a highly-differentiated phenotype and augments cytotoxicity against lymphoma and multiple myeloma target cells. Part II: impact of latent cytomegalovirus infection and catecholamine sensitivity. Brain Behav Immun 49, 59-65 (2015); Bigley, A. B. et al. Acute exercise preferentially redeploys NK-cells with a highly-differentiated phenotype and augments cytotoxicity against lymphoma and multiple myeloma target cells. Brain Behav Immun 39, 160-171 (2014)), which is governed by MR ligand and genetic polymorphisms of the NK-cell repertoire (Stringaris, K. et al. Donor MR Genes 2DL5A, 2DS1 and 3DS1 are associated with a reduced rate of leukemia relapse after HLA-identical sibling stem cell transplantation for acute myeloid leukemia but not other hematologic malignancies. Biol Blood Marrow Transplant 16, 1257-1264 (2010)). CMV IgG serostatus is determined by ELISA. MR genotyping to identify the presence or absence of specific MR genes is determined by polymerase chain reaction following methods previously described (Stringaris, K. et al. Donor MR Genes 2DL5A, 2DS1 and 3DS1 are associated with a reduced rate of leukemia relapse after HLA-identical sibling stem cell transplantation for acute myeloid leukemia but not other hematologic malignancies. Biol Blood Marrow Transplant 16, 1257-1264 (2010)). Haplotype B is assigned if 1 or more MR B defining loci are present (Stringaris, K. et al. Donor MR Genes 2DL5A, 2DS1 and 3DS1 are associated with a reduced rate of leukemia relapse after HLA-identical sibling stem cell transplantation for acute myeloid leukemia but not other hematologic malignancies. Biol Blood Marrow Transplant 16, 1257-1264 (2010)).

Measured Endpoints: The primary endpoints include: (i) changes in PBHC graft composition; (ii) activation of downstream β₂-AR signaling pathways at the protein and mRNA level; (iii) donor comfort; and (iv) donor tolerance to prolonged ISO infusion (up to 4 h), determined as follows:

### (i) Changes in PBHC Graft Composition:

Five-part differential complete blood counts (Beckman Coulter® Ac-T 5diff) and up to 18-color flow cytometry (BD LSRFORTESSA) are used to determine PBMC composition of donor PBMCs. The total number of CD34+ HSCs in whole blood is determined in accordance with the ISHAGE guidelines for simple and standardized enumeration of CD34+ cells (Sutherland, D. R., Anderson, L., Keeney, M., Nayar, R. & Chin-Yee, I. The ISHAGE guidelines for CD34+ cell determination by flow cytometry. International Society of Hematotherapy and Graft Engineering. J Hematother 5, 213-226 (1996)). The ‘favorable’ and ‘unfavorable’ cell types, including their phenotypic identification, are shown in Table 1. Graft composition is quantified in the following ways: (i) the total number of ‘favorable’ and ‘unfavorable’ cell types per ml of blood; and (ii) the total proportion of ‘favorable’ and ‘unfavorable’ cell types in peripheral blood (% of total mononuclear cells). For the experiments involving G-CSF, immune cell subtype numbers are also expressed relative to the total number of CD34+ HSCs in the graft.

(ii) Activation of Downstream β₂-AR Signaling Pathways at the Protein and mRNA Level.

To bolster the in vivo approach, activation of the Gs and β-arrestin pathways are measured ex vivo in response to ISO infusion. Intracellular cAMP and ERK1/2 are measured as indicators of Gs biased signaling and β-arrestin biased signaling in PBMC, respectively. RNA is isolated from PBMC for subsequent qRT-RT-PCR on cAMP and ERK1/2 mRNA. RNA is isolated by Trizol reagent and checked for purity and integrity by standard methods. cAMP and ERK 1/2 standard curves are created by cloned RNA runoffs using TOPO-TA cloning kits (Invitrogen). Template RNA for each gene are serially diluted to create RNA standard curves for qRT-PCR and plotted against each sample to determine RNA quantities.

(iii) Donor Comfort:

It is important to show that the pharmaceutical graft engineering strategy does not elicit undue distress in the donor. A 6-item shortened version of the state subscale of the State-Trait Anxiety Inventory (STAI) that has been validated in the context of blood donation is used (Chell, K., Waller, D. & Masser, B. The Blood Donor Anxiety Scale: a six-item state anxiety measure based on the Spielberger State-Trait Anxiety Inventory. Transfusion 56, 1645-1653 (2016)), asking subjects to complete this before and at 5-minute intervals during the 30-minute infusion trials.

### (iv) Donor Tolerance to Prolonged ISO-Infusion:

Most experimental ISO infusion studies last 15-45 minutes and are well tolerated by healthy participants. All subjects in studies described in Example 1 when ISO was administered without a β₁-AR antagonist intimated that they would be able to tolerate ISO infusion for up to 4 h if required, which is the typical length of time required to collect HSCs from G-CSF mobilized donors by apheresis. However, tachycardia was identified as a potential factor that might limit tolerance to ISO for this length of time. Thus, it is very likely that a pharmaceutical administration strategy designed to block the β₁-AR but not the preferred β₂-AR signaling pathway during ISO infusion will still elicit the desired effects on graft composition (largely β₂-AR mediated) without the undesirable effects of tachycardia and hypertension (largely β₁-AR mediated). The optimal graft engineering strategy identified from the 30-minute infusion trials is applied to a healthy donor population following 5-days of G-CSF treatment. In this experiment, donors receive the required oral pharmaceuticals [(i) bisoprolol alone; (ii) bisoprolol+roflumilast; or (iii) carvedilol] after G-CSF treatment and within a few hours of being infused with ISO continuously for 4 hours at 50 ng/kg/min. Subjects receive daily 5 mcg/kg doses of filgrastim (max dose 480 mcg) (G-CSF), consistent with the procedures used for mobilizing peripheral blood CD34+ HSCs in healthy donors. A resting blood sample is collected (akin to a standard allograft) after G-CSF treatment and prior to oral drug administration and ISO infusion. Blood samples collected every 30-minutes during the infusion are pooled (akin to an apheresis product). Isolated PBMCs from blood collected before and during the 4 h infusion are used for the functional studies described in Example 3. Donor comfort is assessed at 30-minute intervals during the infusion. Donors are allowed to stop and restart the infusion procedure at their own discretion but they are encouraged to tolerate continuous ISO infusion for as long as possible. Tolerance (%) is expressed as the total amount of time ISO was infused relative to the total possible time (4 h).

Statistical Power and Analysis: A sample size of 24 participants is utilized. This sample size has 89% power to detect a large effect (f=0.50, within person correlation=0.5, α=0.05). Hierarchical linear modeling with 3 multi-level models (participant as level-2, time as level-1) is run: intercept-only model (1), means-as-outcome model (2), and the intercepts-and-slopes-as-outcomes model (3). For all models, restricted maximal likelihood estimation is used due to a smaller level-2 sample size, statistical significance is determined by p<0.05, and group means are uncentered. Model 1 allows for a determination between individual variability (level-2) in CMV seropositivity, NK-cell MR gene haplotype, sex, and age, as well as within individual variability (level-1) in donor graft composition. Model 2 determines drug trial [(1) oral placebo+injection placebo (saline); (2) oral placebo+ISO; (3) bisoprolol+ISO; (4) nadolol+ISO; (5) carvedilol+ISO; and bisoprolol+roflumilast+ISO] across the blocks as level-2 predictors of donor graft composition at each time point (0 h, +3 h, +3.5 h). Finally, Model 3 analyzes the cross-level interaction effect of drug and infusion trial on changes in donor graft composition and function over time. Descriptive statistics are used to determine the percentage of donors tolerating ISO infusion for up to 4 h. Statistical significance is set at p<0.05.

### Example 3

### Function of G-CSF Versus G-CSF+ISO Mobilized PBHC Grafts In Vitro and In Vivo.

Blood samples are collected from the G-CSF mobilized participants before and during ISO infusion lasting up to 4 h (as described in Example 2). Complete blood counts are performed prior to the isolation of PBMCs, which are then stored in LN₂ until analysis. The in vitro function of the donor PBHC grafts mobilized with G-CSF+ISO (in combination with the optimal graft engineering strategy identified in Example 2) is compared to G-CSF alone. This is done by measuring: (i) the ex vivo expansion and anti-tumor activity of NK-cells and TCR-γδ cells; (ii) the number of TCR-αβ cells responding to viral antigens; (iii) alloreactivity of TCR-043 T-cells; (iv) the suppressive activity of T_{regs} and MDSCs; and (v) the cytokine profiles of the major lymphocyte subtypes. In addition, the in vivo function of the donor PBHC grafts mobilized with G-CSF+ISO (and the optimal graft engineering strategy identified in Example 2) is compared with G-CSF alone. This is done by transplanting the PBHC grafts to human luciferase labeled tumor (K562 leukemia or SK-N-SH neuroblastoma) bearing and non-tumor bearing NOD-scid-IL2Rγ^{null} (NSG) mice and measuring: (i) engraftment of the human PBHCs, (ii) survival and pathology to xenoGvHD; and (ii) tumor growth and immune cell infiltration. In further experiments, the cell populations in the graft responsible for the beneficial effects in vivo are identified by systematically depleting those cell subsets (e.g. NK-cells, TCR-γδ cells, MDSCs, T_{-regs}, etc) from the graft deemed to be largely affected by ISO (both in terms of composition and function in vitro) prior to transplant in the NSG mice.

Research Design: Scientific Rigor is attained through the use of a repeated measures within-subjects experimental design that allow one to directly compare the function of G-CSF only versus G-CSF+ISO mobilized grafts from the same donor. Samples obtained from subjects in Example 2 are cryopreserved for batch analysis, thus minimizing day to day variability in assay performance. To improve reproducibility, samples collected from a single donor are transplanted to NSG mice in quadruplicate (8 recipient mice per donor, 4×G-CSF only, 4×GCSF+ISO). All endpoint measures in the NSG mice including the pathology assessments will be completed under blinded conditions. Relevant Biological Variables: The in vitro experiments are performed using samples from equal numbers of male and female donors. Equal numbers of male and female mice are used for the xenotransplantation experiments. Measured Endpoints: The following endpoints are measured following (i) the in vitro based assays; and (ii) the in vivo experiments using the NSG mouse model, determined as follows: (i) in vitro based assays:

### NK-Cell Expansion and Cytotoxicity:

NK-cell cytotoxic activity (NKCA) are determined on PBMCs and ‘untouched’ NK-cells magnetically sorted from PBMCs by negative selection (Miltenyi). Standardized allogeneic cell lines derived from AML (HL-60; Kasumi-6), ALL (NALM6, MOLT-4), CML (K562), lymphoma (Daudi, Raji, SU-DHL-10) and neuroblastoma (SK-N-SH; SK-N-DZ), all purchased from ATCC are used as the source of target cells for NKCA assays. A 4-color flow cytometry-based assay, which has been validated against the 51Cr release assay, is used to assess NKCA following published methods (Bigley, A. B. et al. Acute exercise preferentially redeploys NK-cells with a highly-differentiated phenotype and augments cytotoxicity against lymphoma and multiple myeloma target cells. Part II: impact of latent cytomegalovirus infection and catecholamine sensitivity. Brain Behav Immun 49, 59-65 (2015); Bigley, A. B. et al. Latent cytomegalovirus infection enhances anti-tumour cytotoxicity through accumulation of NKG2C+NK cells in healthy humans. Clin Exp Immunol 185, 239-251 (2016)). Changes in the surface expression of activating and inhibitory receptors (NKG2A, NKG2D, NKG2C, NKp30, NKp46, CD158a, CD158b) on NK-cells are determined using flow cytometry, and the phenotypes of the cytotoxic NK-cells are determined by gating on CD107a+ NK-cells following co-culture with target cells. To expand NK-cells ex vivo, isolated NK-cells are stimulated weekly with Clone-9 IL-21 transfected K562 feeder cells, IL-2 and IL-15 for 21-days. Expansion rate is determined by quantifying the number of viable NK-cells in the cultures after 7, 14 and 21-days. NKCA and phenotypic analysis will be repeated after 21-days of culture.

TABLE 3
Viral antigens used for the enumeration of VSTs
(immunodominant antigens highlighted⁷³)

| Virus | Antigen |
|---|---|
| CMV | IE-1, pp65 |
| EBV | EBNA1, LMP2, BMLF1 |
| AdV | Hexon, Penton |
| BK | LT, VP-1 |
| HHV-6 | U14, U90 |

### TCR-γδ Cell Expansion and Cytotoxicity:

TCR-γδ cells will be expanded following a previously described protocol (Kondo, M. et al. Expansion of human peripheral blood gammadelta T cells using zoledronate. Journal of visualized experiments: JoVE (2011)), which involves culturing PBMCs with zoledronic acid and IL-2 for 14-days. This protocol expands Vδ2 TCR-γδ cells with a purity >95%. To expand Vδ1 TCR-γδ cells, isolated TCR-γδ cells are stimulated with PHA and IL-7 for 14-days (Wu, D. et al. Ex vivo expanded human circulating Vdelta1 gammadeltaT cells exhibit favorable therapeutic potential for colon cancer. Oncoimmunology 4, e992749 (2015)). The phenotypic characteristics of the TCR-γδ cells is determined before and after ex vivo expansion. To determine TCR-γδ cell cytotoxicity after 14-days, purified TCR-γδ cells (Vδ1 or Vδ2) are co-cultured with standard allogeneic tumor cell lines following the same procedures described above for NK-cells. The phenotypic characteristics of TCR-γδ cells are determined by flow cytometry before and after ex vivo expansion.

### Viral Specific T-Cells (VSTs):

The total number of VSTs and the number of VSTs responding to each target antigen are determined using pepmixes and IFN-γ ELISPOT assays following procedures described previously (Spielmann, G., Bollard, C. M., Kunz, H., Hanley, P. J. & Simpson, R. J. A single exercise bout enhances the manufacture of viral-specific T-cells from healthy donors: implications for allogeneic adoptive transfer immunotherapy. Scientific reports 6, 25852 (2016)). The number of VSTs per fixed number of PBMCs determined by ELISPOT is adjusted for the total blood T-cell count (VSTs/ml) and relative to the number of CD34+ HSCs in the graft (Spielmann, G, Bollard, C. M., Kunz, H., Hanley, P. J. & Simpson, R. J. A single exercise bout enhances the manufacture of viral-specific T-cells from healthy donors: implications for allogeneic adoptive transfer immunotherapy. Scientific reports 6, 25852 (2016) (Spielmann, G., Bollard, C. M., Kunz, H., Hanley, P. J. & Simpson, R. J. A single exercise bout enhances the manufacture of viral-specific T-cells from healthy donors: implications for allogeneic adoptive transfer immunotherapy. Scientific reports 6, 25852 (2016)). The target antigens used for the enumeration of VSTs are shown in Table 3 and consistent with the major antigens known to cause overt viral disease after alloHCT (Leen, A. M., Heslop, H. E. & Brenner, M. K. Antiviral T-cell therapy. Immunol Rev 258, 12-29 (2014)). Phenotyping of the VSTs is determined using PBMC peptide stimulation, IFN-γ capture assays and flow cytometry (Appay, V., van Lier, R. A., Sallusto, F. & Roederer, M. Phenotype and function of human T lymphocyte subsets: consensus and issues. Cytometry A 73, 975-983 (2008); Maecker, H. T., McCoy, J. P. & Nussenblatt, R. Standardizing immunophenotyping for the Human Immunology Project. Nat Rev Immunol 12, 191-200 (2012)).

### Cytokine Secretion:

The cytokine secretion profile of the major lymphocyte subtypes (CD4+, CD8+, B-cells, NK-cells and TCR-γδ cells) is determined using PMA+ionomycin in the presence of Brefeldin A and intracellular cytokine staining by flow cytometry as described previously (Lavoy, E. C., Bosch, J. A., Lowder, T. W. & Simpson, R. J. Acute aerobic exercise in humans increases cytokine expression in CD27(−) but not CD27(+) CD8(+) T-cells. Brain Behav Immun 27, 54-62 (2013)). The cytokines of interest include: IFN-γ, TNF-α, IL-2, IL-4, IL-7, IL-10, IL-15, IL-17.

### MDSC and T_{-reg} Suppression:

The ability of MDSCs (monocytic and granulocytic) and T_{-regs} to suppress the proliferation of CD3/CD28 stimulated T-cells in vitro is compared between those cells isolated from G-CSF only versus G-CSF+ISO mobilized PBHC grafts using CellTrace™ assays and flow cytometry as described previously (Janikashvili, N. et al. Allogeneic effector/memory Th-1 cells impair FoxP3+ regulatory T lymphocytes and synergize with chaperone-rich cell lysate vaccine to treat leukemia. Blood 117, 1555-1564 (2011); Alizadeh, D. et al. Doxorubicin eliminates myeloid-derived suppressor cells and enhances the efficacy of adoptive T-cell transfer in breast cancer. Cancer Res 74, 104-118 (2014)).

(ii) in vivo experiments using the NOD-scid-IL2Rγ^{null} (NSG) humanized mouse model:

### Engraftment and Xeno-GvHD:

To compare the likelihood of the PBHC grafts mobilized with G-CSF alone or G-CSF+ISO, (±directed Gs or β-arrestin signaling as guided from Example 2) to cause GvHD, PBHC grafts are thawed from cryopreservation, phenotyped, checked for viability, washed, and resuspended in normal saline. 10×10⁶ viable cells are injected into the tail vein of non-tumor bearing, irradiated (200 cGy for 24 h) 8-10 wk old NSG mice. For human cell engraftment, percentages of human immune cells and mouse immune cells is determined on a weekly basis from mouse blood (0.2-0.5 ml) collected from a submandibular vein. Blood is labeled with both mouse (CD45) and human antibodies (CD45, CD3, CD8, CD4, CD56, CD20) to determine percent chimerism and the composition of lymphocyte subsets (e.g. T-cells, CD4+ T-cells, CD8+ T-cells, B-cells, TCR-γδ cells, NK-cells) among the Human CD45+ cells. Subpopulations of these broad lymphocyte subsets found to be affected by ISO (e.g. naïve, EM, CM, EMRA, NKT, iNKT, T_{regs}) are enumerated at weekly intervals to check for persistence in the host. Xeno-GvHD is determined twice weekly using a previously determined scoring system that takes into account weight loss, hair loss, rough coat, abnormal posture, diarrhea, changes in respiration, and changes in activity level (Gu, G., Yang, J. Z. & Sun, L. X. Correlation of graft immune composition with outcomes after allogeneic stem cell transplantation: Moving towards a perfect transplant. Cell Immunol 323, 1-8 (2018)). Upon sacrifice or succumbing to disease, GvHD target organs (lungs, liver, skin, and intestine) are removed. Half the organ is fixed in 10% formalin and embedded in paraffin, sectioned and stained with hematoxylin and eosin for pathohistology. The other half of each organ is enzymatically digested to a single cell suspension (SCS) and infiltrating immune cells are enumerated and phenotyped by flow cytometry. Kaplan-Meier survival curves are generated for the groups receiving PBHC grafts mobilized with G-CSF only and those G-CSG+ISO grafts, stratified by the sex of the mouse.

### Graft-Versus-Tumor (GvT) Effect:

Irradiated NSG mice are engrafted with luciferase tagged human leukemia (K562) or neuroblastoma (SK-N-SH) cell lines and transplanted with PBHC grafts mobilized with G-CSF only or G-CSF+ISO, as described above. The K562 cell line is administered via the tail vein and the SK-N-SH will be injected subcutaneously. The effects of the graft on both a liquid and solid tumor are tested; the neuroblastoma cell line was selected because it is considered an NK-cell and TCR-γδ cell sensitive tumor and allows one determine the number of tumor infiltrates after transplant (Duan, D. S., Farmer, D., Rayner, A. A. & Sadee, W. Cytotoxicity of lymphokine-activated killer cells against human neuroblastoma cells: modulation by neuroblast differentiation. Medical and pediatric oncology 18, 339-344 (1990); Pressey, J. G. et al. In vivo expansion and activation of gammadelta T cells as immunotherapy for refractory neuroblastoma: A phase 1 study. Medicine 95, e4909 (2016); Sartelet, H., Durrieu, L., Fontaine, F., Nyalendo, C. & Haddad, E. Description of a new xenograft model of metastatic neuroblastoma using NOD/SCID/Il2rg null (NSG) mice. In vivo (Athens, Greece) 26, 19-29 (2012)). Tumor growth is measured non-lethally on a weekly basis by bioluminescence imaging. Upon sacrifice or succumbing to disease, neuroblastoma tumors are excised and weighed to determine tumor weight to body weight ratios. Half the tumor is sectioned and stained for pathohistology while the other half is used to identify infiltrating human immune cells by immunohistochemistry.

Statistical Power and Analysis: Based on the available human subjects (n=24), power set to 80%, and an error probability α=0.05, the in vitro aim is able to detect a large effect (f=0.52). For the in vitro experiments, graft function is compared between the G-CSF only and G-CSF+ISO mobilized PBHC grafts using paired sample t-tests. Significance in difference between the two dependent means is determined by a one-tailed (p<0.025) critical t=1.71 for a large effect size (f=0.05, α=0.05). Specifically, paired mean differences in the following outcome measures are examined: (1) NK-cell expansion and cytotoxicity, (2) TCR-γδ cell expansion and cytotoxicity, (3) VSTs, (4) cytokine secretion, and (5) MDSC and T_{-reg} suppression. Based on data showing a large effect of PBMC on aGHVD and survival (Tobin, L. M., Healy, M. E., English, K. & Mahon, B. P. Human mesenchymal stem cells suppress donor CD4(+) T cell proliferation and reduce pathology in a humanized mouse model of acute graft-versus-host disease. Clin Exp Immunol 172, 333-348 (2013)), the in vivo aim has been powered at 80% to detect a large effect (f=0.50, α=0.05), yielding a sample size of 34 NSG mice. For the in vivo experiments, to determine survivability percentages, Kaplain-Meier curves are analyzed. To determine the effects of PBHC grafts mobilized with G-CSF+ISO (±directed Gs or β-arrestin signaling as guided from Example 2) on engraftment, xeno-GvHD, and GvT, a maximum-likelihood random intercepts linear mixed model (LMM) are specified that includes a group effect (with ISO or without) and a relevant biological variable effect (CMV serostatus [positive,negative] and MR gene haplotype [expressed, not expressed]). The residual matrix is structured to account for correlation over G-CSF only and G-CSF+ISO within individuals (i.e. repeated measures). An a priori specified contrast tests the interaction effect of G-CSF+ISO and CMV serostatus and MR gene haplotype on (1) engraftment, (2) xeno-GvHD, and (3) GvT. Significance will be set at p<0.05.

### Example 4

### Allogeneic Transplantation of G-CSF+ISO Mobilized PBHC Grafts

24 patients and a comparable number of controls receiving allogeneic PBHC grafts from matched related donors (MRDs) are enrolled. It is determined if alloHCT using PBHC grafts mobilized with G-CSF+ISO in MRDs is both feasible and safe. Feasibility is determined by the percentage of donor patient dyads from all eligible patients electing to participate in this study without attrition. Safety is determined from the standpoint of both the donor and the patient. Depending on the results from Example 2, all donors receive one of three oral drug administration strategies (1. bisoprolol only, 2. bisprolol+roflumilast, or 3. carvedilol) as a single dose within 3 h prior to being infused with ISO during PBHC collections by apheresis. The goal is to infuse ISO for the entire PBHC collection procedure (˜3-4 hours). State level anxiety is determined every 30-minutes during apheresis and ISO infusion ceases if the donor is deemed to be unduly stressed. Donors are excluded from the treatment arm of the study (and also excluded as controls) if ISO is delivered for less than 50% of the total PBHC collection time. For the patients, the primary safety endpoints are engraftment, incidence and severity of acute and chronic GvHD, viral reactivation, and non-relapse mortality. Patients receive one of two standard myeloablative conditioning regimens based on disease (cyclophosphamide and total body irradiation or Busulfan Fludalabine and Melphalan) followed by PBHCT.

It is further determined if clinical outcomes in the patients (e.g. GvHD, viral reactivation) correlate with the composition of immune cell subtypes in the PBHC grafts. Outcome Measures: These include (i) the percentage of donor/patient dyads enrolling and completing the study, and (ii) the percentage of donors in the treatment arm tolerating ISO infusion >50% of the total PBHC collection time. Measured clinical endpoints include (i) time to myeloid, erythroid and platelet engraftment, (ii) incidence and severity of acute and chronic GvHD, (iii) viral reactivation by weekly quantitative PCR assays (CMV, EBV, adenovirus, HHV6, BK virus) and (iv) immune reconstitution post alloHCT (described in detail below).

### Immune Reconstitution:

This is one of the primary prognostic factors affecting outcomes following alloHCT as it has an impact on infection, GvHD and relapse. T-cell reconstitution is determined in all patients (treatment arm and control). It is examined whether the reconstituting T-cell subsets differ following G-CSF+ISO (in conjunction with bisoprolol, bisoprolol+roflumilast or carvedilol) compared to G-CSF alone. Immunophenotyping of peripheral blood is performed by multiparameter flow cytometry on days +28, +56, +100, and +180. The proportion of CD4⁺ and CD8⁺ T_{N} cells is determined and T_{N} that are recent thymic emigrants (RTEs) is defined by co-expression of CD31 (Kohler, S. & Thiel, A. Life after the thymus: CD31+ and CD31− human naive CD4+ T-cell subsets. Blood 113, 769-774 (2009)). The frequencies of T_{SCM}, T_{CM}, T_{EM}, and T_{EMRA} is determined as well (phenotypically as defined in Example 2) (Appay, V., van Lier, R. A., Sallusto, F. & Roederer, M. Phenotype and function of human T lymphocyte subsets: consensus and issues. Cytometry A 73, 975-983 (2008); Maecker, H. T., McCoy, J. P. & Nussenblatt, R. Standardizing immunophenotyping for the Human Immunology Project. Nat Rev Immunol 12, 191-200 (2012)). Although TCR-γδ cells represent only 5% of T-cells, they are thought to participate in the regulation of innate and adaptive immune responses, facilitating engraftment and exerting GvT and antiviral activity without exacerbating GvHD (Perko, R. et al. Gamma delta T cell reconstitution is associated with fewer infections and improved event-free survival after hematopoietic stem cell transplantation for pediatric leukemia. Biol Blood Marrow Transplant 21, 130-136 (2015)). The reconstitution of Vδ1 and Vδ2 TCR-γδ cells and their phenotypic characteristics in the patients is determined following the procedures described for characterizing these cells in donor PBHC grafts in Example 2. NK cells have anti-viral and GvT effects and are evaluated by CD3, CD16 and CD56 expression and cytotoxicity assays against NK sensitive targets, such as K562, as previously published (Katsanis, E. et al. Proliferation and cytolytic function of anti-CD3+interleukin-2 stimulated peripheral blood mononuclear cells following bone marrow transplantation. Blood 78, 1286-1291 (1991); Katsanis, E. et al. Infusions of interleukin-1 alpha after autologous transplantation for Hodgkin's disease and non-Hodgkin's lymphoma induce effector cells with antilymphoma cytolytic activity. J Clin Immunol 14, 205-211 (1994)). NKT cells represent a type of T-cell with NK-cell characteristics. Early reconstitution of NK-cells, particularly the immature CD56^{high} subset, and NKT cells may be associated with decreased GvHD and increased survival (Pillai, A. B., George, T. I., Dutt, S., Teo, P. & Strober, S. Host NKT cells can prevent graft-versus-host disease and permit graft antitumor activity after bone marrow transplantation. J Immunol 178, 6242-6251 (2007); Ullrich, E. et al. Relation between Acute GVHD and NK Cell Subset Reconstitution Following Allogeneic Stem Cell Transplantation. Frontiers in immunology 7, 595 (2016)). NKT (CD1d⁺CD3⁺CD56⁺CD161⁺), immature NK (CD3⁻CD56⁺CD14⁻CD3⁻CD56^{hi}CD16^{dim}) and mature cytotoxic NK (CD3⁻CD56⁺CD14⁻CD3⁻CD56^{dim}CD16^{high}) cell reconstitution is examined by flow cytometry (Rubio, M. T. et al. Early posttransplantation donor-derived invariant natural killer T-cell recovery predicts the occurrence of acute graft-versus-host disease and overall survival. Blood 120, 2144-2154 (2012)).

All publications and patents mentioned in the above specification are herein incorporated by reference. Various modifications and variations of the described method and system of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled relevant fields are intended to be within the scope of the following claims.

1. A method of obtaining stem cells from a donor, comprising:
a) administering a stem cell mobilization agent, a β-adrenergic receptor agonist (β-AR), and at least one additional agent selected from the group consisting of a phosphodiesterase-4 (PDE-4) inhibitor, and a β-AR antagonist; and
b) isolating said stem cells from said donor.

2. The method of claim 1, wherein said donor is a human.

3. The method of claim 1, wherein said stem cells are hematopoietic stem cells.

4. The method of claim 1, wherein said stem cells are peripheral blood hematopoietic cells.

5. The method of claim 1, wherein said β-AR is selected from the group consisting of isoproterenol, denopamine, dobutamine, dopexamine, prenalterol, and xamoterol.

6. The method of claim 5, wherein said β-AR is isoproterenol.

7. The method of claim 1, wherein said PDE-4 inhibitor is selected from the group consisting of roflumilast, apremilast, cilomilast, crisaborole, diazepam, ibudilast, luteolin, mesembrenone, piclamilast, and rolipram.

8. The method of claim 7, wherein said PDE-4 inhibitor is roflumilast.

9. The method of claim 1, wherein said β-AR antagonist is a β₁-AR antagonist.

10. The method of claim 9, wherein said β₁-AR antagonist is selected from the group consisting of bisoprolol, acebutolol, atenolol, betaxolol, celiprolol, metoprolol, nebivolol, and esmolol.

11. The method of claim 10, wherein said β₁-AR antagonist is bisoprolol.

12. The method of claim 1, wherein said additional agent is a PDE-4 inhibitor and a β₁-AR antagonist.

13. The method of claim 1, wherein said β₁-AR antagonist and said PDE-4 inhibitor are administered orally.

14. The method of claim 13, wherein said β₁-AR antagonist and said PDE-4 inhibitor are administered 3 hours prior to said infusion of said β-AR.

15. The method of claim 1, wherein said β-AR is administered via infusion for a duration of approximately 4 hours.

16. The method of claim 1, wherein said isolating said stem cells occurs immediately following said administration of said β-AR.

17. The method of claim 1, wherein said stem cell mobilization agent is selected from the group consisting of G-CSF, GM-CSF, and plerixafor.

18. The method of claim 1, wherein said stem cells exhibit one or more properties selected from the group consisting of increased graft-versus tumor effect, decreased viral reactivation, reduced graft-versus-host-disease, and reduced lymphoproliferative disease relative to stem cells mobilized by stem cell mobilization agent alone.

19. Stem cells isolated by the method of claim 1.

20-26. (canceled)

27. A method of treating a hematological malignancy or immune system disorder, comprising:
administering the stem cells of claim 19 to a subject in need thereof.

28-32. (canceled)
